(19) United States
(12) Patent Application Publication
(10) Pub. No.: US 2009/0275998 A1
(43) Pub. Date: Nov. 5, 2009

(54) EXTRA-CARDIAC IMPLANTABLE DEVICE WITH FUSION PACING CAPABILITY

(75) Inventors: John E. Burnes, Coon Rapids, MN (US); Becky Lynn Dolan, Chisago City, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/432,502

(22) Filed: Apr. 29, 2009

Related U.S. Application Data
(63) Continuation-in-part of application No. 12/112,833, filed on Apr. 30, 2008.

(51) Int. Cl. A61N 1/365; A61N 1/39

(52) U.S. Cl. 607/4; 607/17

(57) ABSTRACT

According to this disclosure, a non-transvenous pacing and, optionally defibrillation, therapy device is implanted subcutaneously and oriented to provide cardiac sensing from electrodes spaced from a heart and deliver pacing and/or defibrillation from one or more non-transvenous electrodes (e.g., an epicardial or pericardial electrode or electrode patch). A subject receiving a device according to this disclosure is monitored to confirm a relatively stable bundle branch block (i.e., delayed activation) of one ventricle. The subcutaneous device has electrodes disposed on the housing and/or having an electrode on a subcutaneous medical lead is oriented so that the pacing (and sensing) vector impinges mainly upon the one ventricle, and/or optionally an epicardial or pericardial lead is deployed to a last-to-depolarize ventricle (e.g., a left ventricle) so that single-ventricular pacing is delivered to achieve fusion depolarization of both ventricles.

### CROSS REFERENCE AND STATEMENT OF INCORPORATION

This patent application is a continuation-in-part of previously filed co-pending application Ser. No. 12/112,833, filed Apr. 30, 2008, the entire contents of which is incorporated by reference herein. This patent application also relates to co-pending application Ser. No. 11/343,677 filed 31 Jan. 2006 entitled, “SUBCUTANEOUS ICD WITH SEPARATE CARDIAC RHYTHM SENSOR,” the entire contents of which is also incorporated by reference herein.

### FIELD

The disclosure pertains to cardiac resynchronization therapy (CRT) delivery pacing systems that deliver fusion-based CRT via ventricular pre-excitation.

### BACKGROUND

It has been shown that in certain patients exhibiting symptoms resulting from congestive heart failure (CHF), cardiac output is enhanced by timing the delivery of a left ventricular (LV) pacing pulse, typically via a lead disposed in a portion of the great cardiac vein to evoke a depolarization of the LV in fusion with the intrinsic depolarization of the right ventricle (RV). The fusion depolarization enhances stroke volume in those hearts in which the RV depolarizes first due to intact atrio-ventricular (AV) conduction, but wherein the AV conducted depolarization of the LV is unduly delayed. The fusion depolarization of the LV is attained by timing the delivery of the LV pace (LVp) pulse to follow the intrinsic depolarization of the RV but to precede the intrinsic depolarization of the LV.

However, due to a number of factors related to the complexity of typical CRT pacers and particularly to the placement of multiple transvenous leads, current CRT systems may not always effectively deliver CRT. The cost and complexity of programming and implanting triple-chamber devices can also pose a barrier to some patients obtaining chronic CRT delivery.

A need therefore exists in the art to simply, efficiently and chronically deliver CRT to patients suffering from various cardiac conduction abnormalities who might not otherwise receive the benefits of CRT therapy.

### SUMMARY

According to this disclosure, a non-transvenous pacing and, optionally defibrillation device is implanted subcutaneously and oriented to provide pacing therapy from non-transvenous electrodes using leads located exterior to the heart. “Non-transvenous” electrodes include electrodes that are implanted without the need to pas electrode-bearing leads through the vasculature and into the heart. Such leads may include, for example, subcutaneous, pericardial, epicardial and/or myocardial electrodes of any type known to the art. A subject receiving a device according to this disclosure is monitored to confirm a relatively stable bundle branch block or delayed activation of one ventricle. The subcutaneous device having electrodes disposed on the housing and/or having an electrode on a subcutaneous medical lead is oriented so that the pacing vector impinges mainly upon the one ventricle. A preferred mechanism to accomplish this result comprises placement of an electrode on or adjacent the pericardium or epicardium or in the myocardium of the ventricle to be stimulated.

A single pacing stimulus is then delivered upon expiration of an AV interval timed from at least one prior intrinsic atrial event, represented herein as “As” determined from at least one prior “As” that resulted in an intrinsic sensed ventricular event (Vs). The triggering event, As, can emanate from the right atrium (RA) or the left atrium (LA) and the “single” ventricular pacing stimulus is timed to pre-excite the one ventricle so that intra-ventricular mechanical synchrony results. The mechanical synchrony results from the fusing of the two ventricular depolarization wave fronts (i.e., one “paced” and the other more or less intrinsically-conducted). Accordingly, delivery of a single “ventricular” pacing stimulus occurs upon expiration of a fusion-AV or, herein referred to as the pre-excitation interval (“PEI”). One way to express this relationship defines the PEI as being based on an intrinsic AV interval or intervals from an immediately prior cardiac cycle or cycles. Thus, the PEI can be expressed as PEI=AVₙ₋₁−Vₚₑᵢ, wherein the AV interval represents the interval from an A-event (As) to the resulting intrinsic depolarization of a ventricle (for a prior cardiac cycle) and the value of PEI equals the desired amount of pre-excitation needed to effect ventricular fusion (expressed in ms). For a patient with LBBB conduction status (for a current cardiac cycle “n”) the above formula can be expressed as: A-LVpₙ=A-RVₙ₋₁−LVₚₑᵢ and for a patient suffering from RBBB conduction status the formula reduces to: A-RVpₙ=A-LVₙ₋₁−RVₚₑᵢ.

As noted above, the timing of the single pacing stimulus is an important parameter when delivering therapy according to the foregoing. While a the interval between a single, immediately prior atrial event to a sensed ventricular depolarization can be utilized to set the PEI and derive the timing for delivering pacing, more than a single prior sensed AV interval, a prior PEI, a plurality of prior sensed AV intervals or prior PEIs can be utilized (e.g., mathematically calculated values such as a temporal derived value, a mean value, an averaged value, a median value and the like). Also, a time-weighted value of the foregoing can be employed wherein the most recent values receive additional weight. Alternatively, the PEI can be based upon heart rate (HR), a derived value combining HR with an activity sensor input, P-wave to P-wave timing, R-wave to R-wave timing and the like. Again, these values may be time-weighted in favor of the most, or more, recent events. Of course, other predictive algorithms could be used which would account for variability, slope or trend in AV interval timing and thereby predict AV characteristics.

Among other aspects, this provides an energy-efficient manner of providing single ventricle, pre-excitation fusion-pacing therapy delivered from a non-transvenously implanted medical device generally. A non-contacting (e.g. subcutaneous) electrode pair, for example as disclosed in US Patent Application Publication No. US 2006/0122649 A1 by Ghanem, et al., incorporated herein by reference in its entirety may also be used to practice the invention. Other non-transvenous electrode configurations which deliver a pacing stimulus which can be directed to stimulate a desired ventricle can also be employed, including electrodes associated with a stimulation pulse generator located an or adjacent the outer wall of the heart, as disclosed in U.S. Pat. No. 5,814,089, issued to Stokes, et al. and incorporated herein by reference in its entirety.

In one preferred embodiment, a single epicardial, pericardial or myocardial pacing electrode or electrode pair is deployed to contact with the last-to-depolarize ventricle. The implant procedure for an extra-cardiac ICD (e.g. sub-Q or sub-muscular) used to practice the foregoing—in the pectoral region or infra-clavicular region, in conjunction with the present invention, allows for chronic application of CRT. Preexisting implant leads and tools have been developed that make transcutaneous implants of such electrodes feasible using a sub-xiphoid or infra-clavicular approach. For example U.S. Pat. No. 3,737,579, issued to Bolduc and U.S. Pat. No. 4,010,758, issued to Rockland, et al., both incorporated herein by reference in their entireties disclose such devices. The challenge with regard to obtaining CRT by simply placing an electrode or electrode pair configured to stimulate a single ventricle is that proper CRT can only be delivered if the ventricular pacing pulse is timed to the intrinsic atrial activity and/or the activity of the other ventricle.

A preferred embodiment disclosed herein comprises a single lead extra-vascular system, with the lead electrode or electrodes placed on the LV. Fusion based CRT may be delivered in response to by far-field sensed atrial and ventricular signals (P-waves and R-waves). Fusion pacing algorithms whose basic concepts have already been disclosed could then be applied, for example by monitoring intrinsic A-V intervals and pacing the LV at shorter A-VP intervals. A triggered pacing mode could be a backup mode to fusion if reliable P-waves cannot be detected, triggering an LV pace off of a far-field ventricular sense. Special signal processing (e.g., filtering) techniques are used to accurately determine the P-wave, including detecting an R-wave and then looking back over a window where the P-wave is detected. Additional electrode/lead locations may be used to pick up P-wave and R-wave activity, including extra-vascular leads passing around the chest to the posterior of a subject.

Some features of the disclosure may include: delivery of fusion pacing to a single ventricle using non-transvenously implanted electrodes and far-field sensing of the P-waves; computation of a Fusion A-V pacing interval (P-AV delay) al based on a periodic evaluation of a sensed A-V interval and the use of a pre-excitation interval (PEI); and/or titration of the PEI or detection point on the P-wave based on the resulting paced fusion beat; and a switch from Fusion to triggered pacing if P-waves cannot be reliably detected. The lead attached to the heart may optionally include defibrillation coils to assist in lowering DFTs. Addition of a second non-transvenous lead to deliver biventricular (BiV) pacing is also an option. Substitution of a remote-controlled stimulator mounted to the outer wall of a ventricle for the non-transvenous lead or leads is also possible.

The foregoing and other aspects and features of the present disclosure will be more readily understood from the following detailed description of the embodiments thereof, when considered in conjunction with the drawings which like reference numerals indicate similar structures throughout the several views, and with reference to the claims appearing at the end of this disclosure.

### BRIEF DESCRIPTION OF THE DRAWINGS

FIG. 1 is a schematic diagram of an exemplary subcutaneous device in which the present disclosure may be usefully practiced.

FIG. 2 is a perspective view of a system according to certain embodiments of the disclosure.

FIG. 3A is an exemplary schematic diagram of electronic circuitry within a hermetically sealed housing of a subcutaneous device of the present disclosure.

FIG. 3B is a schematic diagram of signal processing aspects of a subcutaneous device according to an exemplary embodiment of the present disclosure.

FIG. 3C illustrates exemplary subcutaneous and filtered electrogram signals as employed by an exemplary embodiment of the present disclosure.

FIG. 4 illustrates an embodiment of the energy efficient, single-pacing stimulus, ventricular pre-excitation pacing mode according to the present disclosure.

FIG. 5 depicts a process for periodically ceasing delivery of the pre-excitation, single ventricular pacing therapy to determine the cardiac conduction status of a patient and performing steps based on the status.

FIG. 6 illustrates an alternative embodiment to the invention as illustrated in FIGS. 1 and 2.

FIG. 7 illustrates a second alternative embodiment to the invention as illustrated in FIGS. 1 and 2.

### DETAILED DESCRIPTION

In the following detailed description, references are made to illustrative embodiments for carrying out an energy-efficient, single-pacing stimulus, ventricular pre-excitation pacing mode according to the present disclosure. It is understood that other embodiments may be utilized without departing from the scope of the disclosure. For example, examples are disclosed in detail herein in the context of an intrinsically-based or AV sequential (evoked) uni-ventricular pacing system with remote ventricular sensing. This provides an efficient pacing modality for restoring electromechanical ventricular synchrony based upon either atrial-paced or atrial-sensed events particularly for patients with some degree of either chronic, acute or paroxysmal ventricular conduction block (e.g., intraventricular, LBBB, RBBB). A system according to the disclosure efficiently can provide cardiac resynchronization therapy (CRT) with a single pacing stimulus per cardiac cycle.

The following issued U.S. patents are hereby incorporated into this disclosure as if fully set forth herein; namely, U.S. Pat. No. 6,871,096 to Hill, entitled, “System and Method for Bi-Ventricular Fusion-pacing;” issued U.S. Pat. No. 7,254,442 to Pilmeyer and van Gelder entitled, “APPARATUS AND METHODS FOR ‘LEPARS’ INTERVAL-BASED FUSION-PACING;” and U.S. Pat No. 7,181,284 to Burnes and Mullen entitled, “APPARATUS AND METHODS OF ENERGY EFFICIENT, ATRIAL-BASED BI-VENTRICULAR FUSION-PACING.”

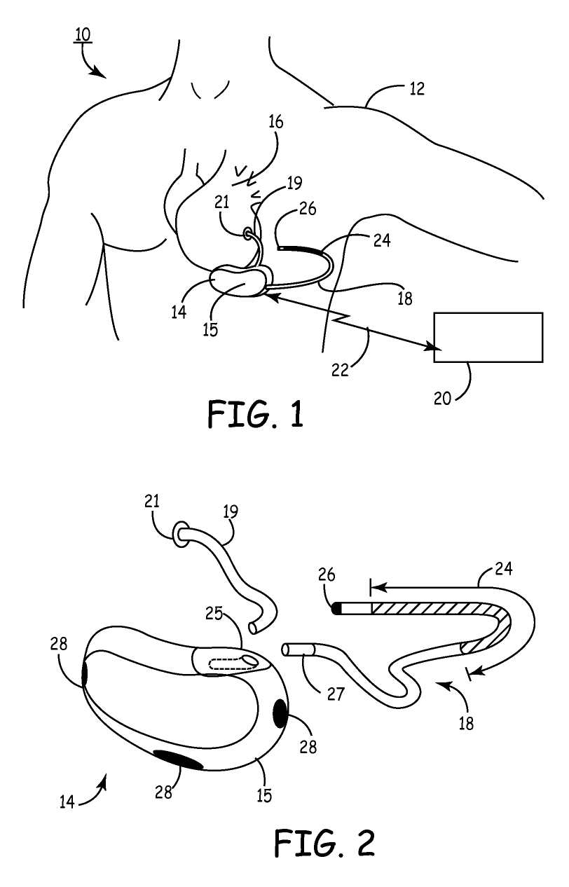

FIG. 1 is a schematic diagram of an exemplary device in which the present disclosure may be usefully practiced. As illustrated in FIG. 1, a device 14 according to an embodiment of the present disclosure is subcutaneously implanted outside the ribcage of a patient 12, anterior to the cardiac notch. Further, a subcutaneous sensing and cardioversion/defibrillation therapy delivery lead 18 in electrical communication with subcutaneous device 14 is tunneled subcutaneously into a location adjacent to a portion of a latissimus dorsi muscle of patient 12. Specifically, lead 18 is tunneled subcutaneously from the median implant pocket of the subcutaneous device 14 laterally and posterior toward the patient's back to a location opposite the heart such that the heart 16 is disposed between the subcutaneous device 14 and the distal electrode coil 24 and distal sensing electrode 26 of lead 18. Lead 19, carrying electrode 21 may be any known type of epicardial or myocardial electrode bearing lead known to the art. Electrode 21 is illustrated as located on the patient's left ventricle.

It is understood that while the subcutaneous device 14 is shown positioned through loose connective tissue between the skin and muscle layer of the patient, the term “subcutaneous device” is intended to include a device that can be positioned in the patient to be implanted using any non-intravenous location of the patient, such as below the muscle layer or within the thoracic cavity, for example.

Further referring to FIG. 1, a programmer 20 is shown in telemetric communication with subcutaneous device 14 by an RF communication link 22. Communication link 22 may be any appropriate RF link such as Bluetooth, WiFi, MICS, or as described in U.S. Pat. No. 5,683,432 “Adaptive Performance-Optimizing Communication System for Communicating with an Implantable Medical Device” to Goedeke, et al. and incorporated herein by reference in its entirety.

Subcutaneous device 14 includes a housing 15 that may be constructed of stainless steel, titanium or ceramic as described in U.S. Pat. Nos. 4,180,078 “Lead Connector for a Body Implantable Stimulator” to Anderson and 5,470,345 “Implantable Medical Device with Multi-layered Ceramic Enclosure” to Hassler, et al, both incorporated herein by reference in their entireties. The electronics circuitry of SubQ ICD 14 may be incorporated on a polyimide flex circuit, printed circuit board (PCB) or ceramic substrate with integrated circuits packaged in leadless chip carriers and/or chip scale packaging (CSP).

Optional subcutaneous lead 18 as illustrated includes a defibrillation coil electrode 24, a distal sensing electrode 26, an insulated flexible lead body and a proximal connector pin 27 (shown in FIG. 2) for connection to the housing 15 of the subcutaneous device 14 via a connector 25. In addition, one or more electrodes 28 (shown in FIG. 2) are positioned along the outer surface of the housing to form a housing-based subcutaneous electrode array (SEA). Distal sensing electrode 26 is sized appropriately to match the sensing impedance of the housing-based subcutaneous electrode array.

It is understood that while device 14 is shown with electrodes 28 positioned on housing 15, according to an embodiment of the present disclosure electrodes 28 may be alternatively positioned along one or more separate leads connected to device 14 via connector 25. Atrial sensing is accomplished via the subcutaneously located electrodes. Ventricular sensing may be accomplished using any of the electrodes, including the subcutaneous electrodes and/or electrodes located on or adjacent the heart as described below.

Continuing with FIG. 2, electrodes 28 are welded into place on the flattened periphery of the housing 15. In the embodiment depicted in this figure, the complete periphery of the SubQ ICD may be manufactured to have a slightly flattened perspective with rounded edges to accommodate the placement of the electrodes 28. The electrodes 28 are welded to housing 15 (to preserve hermaticity) and are connected via wires (not shown) to electronic circuitry (described herein below) inside housing 15. Electrodes 28 may be constructed of flat plates, or alternatively, may be spiral electrodes as described in U.S. Pat. No. 6,512,940 “Subcutaneous Spiral Electrode for Sensing Electrical Signals of the Heart” to Brabec, et al. and mounted in a non-conductive surround shroud as described in U.S. Pat. Nos. 6,522,915 “Surround Shroud Connector and Electrode Housings for a Subcutaneous Electrode Array and Leadless ECGs” to Ceballos, et al. and 6,622,046 “Subcutaneous Sensing Feedthrough/Electrode Assembly” to Fraley, et al, all incorporated herein by reference in their entireties. The electrodes 28 of FIG. 2 can be positioned to form orthogonal or equilateral signal vectors, for example.

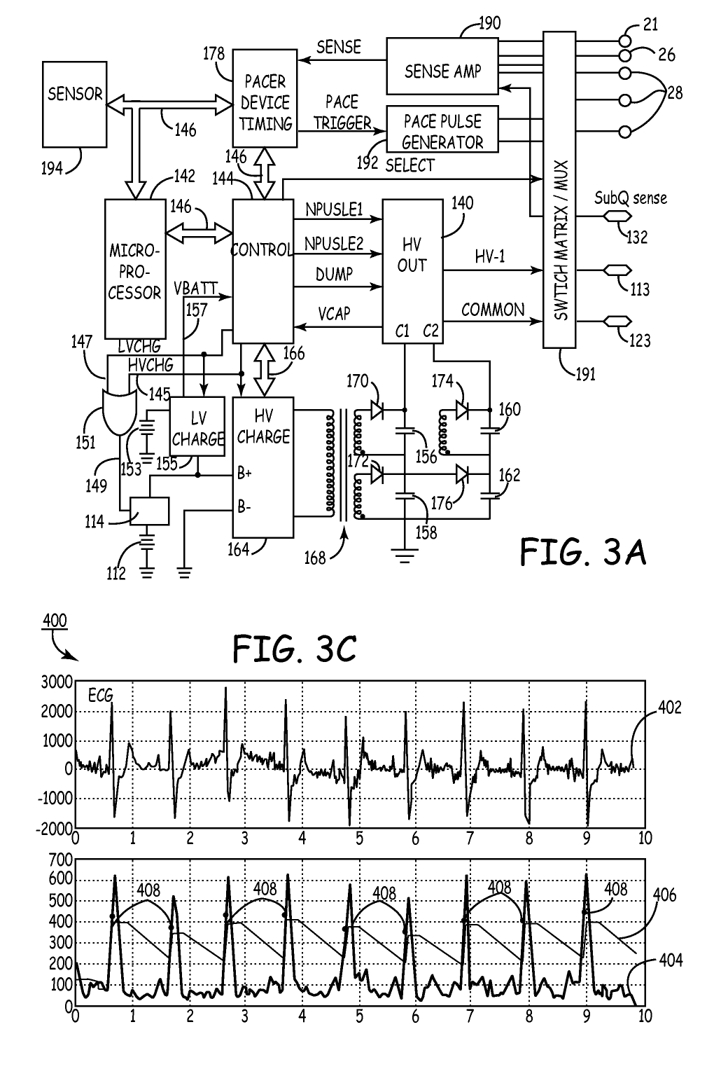

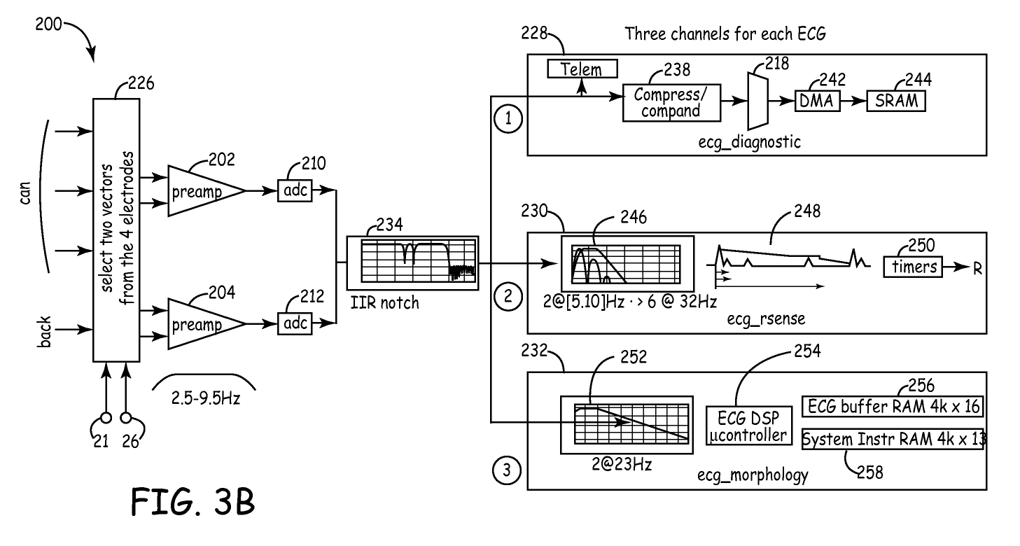

The electronic circuitry employed in subcutaneous device 14 can take any of the known forms that detect a tachyarrhythmia from the sensed ECG and provide cardioversion/defibrillation shocks as well as post-shock pacing as needed while the heart recovers. A simplified block diagram of such circuitry adapted to function employing the first and second cardioversion-defibrillation electrodes as well as the ECG sensing and pacing electrodes described herein below is set forth in FIG. 3A. It will be understood that the simplified block diagram does not show all of the conventional components and circuitry of such devices including digital clocks and clock lines, low voltage power supply and supply lines for powering the circuits and providing pacing pulses or telemetry circuits for telemetry transmissions between the device 14 and external programmer 20.

FIG. 3A is an exemplary schematic diagram of electronic circuitry within a hermetically sealed housing of a subcutaneous device according to an embodiment of the present disclosure. As illustrated in FIG. 3A, subcutaneous device 14 includes a low voltage battery 153 coupled to a power supply (not shown) that supplies power to the circuitry of the subcutaneous device 14 and the pacing output capacitors to supply pacing energy in a manner well known in the art. The low voltage battery 153 may be formed of one or two conventional LiCFₓ, LiMnO₂ or Lil₂ cells, for example. The subcutaneous device 14 also includes a high voltage battery 112 that may be formed of one or two conventional LiSVO or LiMnO₂ cells. Although two both low voltage battery and a high voltage battery are shown in FIG. 3A, according to an embodiment of the present disclosure, the device 14 could utilize a single battery for both high and low voltage uses.

Further referring to FIG. 3A, subcutaneous device 14 functions are controlled by means of software, firmware and hardware that cooperatively monitor the ECG, determine when a cardioversion-defibrillation shock or pacing is necessary, and deliver prescribed cardioversion-defibrillation and pacing therapies. The subcutaneous device 14 may incorporate circuitry set forth in commonly assigned U.S. Pat. Nos. 5,163,427 “Apparatus for Delivering Single and Multiple Cardioversion and Defibrillation Pulses” to Keimel and 5,188,105 “Apparatus and Method for Treating a Tachyarrhythmia” to Keimel for selectively delivering single phase, simultaneous biphasic and sequential biphasic cardioversion-defibrillation shocks typically employing ICD IPG housing electrodes 28 coupled to the COMMON output 123 of high voltage output circuit 140 and cardioversion-defibrillation electrode 24 disposed posteriorly and subcutaneously and coupled to the HVI output 113 of the high voltage output circuit 140. Outputs 132 of FIG. 3A is coupled to sense electrode 26.

The cardioversion-defibrillation shock energy and capacitor charge voltages can be intermediate to those supplied by ICDs having at least one cardioversion-defibrillation electrode in contact with the heart and most AEDs having cardioversion-defibrillation electrodes in contact with the skin. The typical maximum voltage necessary for ICDs using most biphasic waveforms is approximately 750 Volts with an associated maximum energy of approximately 40 Joules. The typical maximum voltage necessary for AEDs is approximately 2000-5000 Volts with an associated maximum energy of approximately 200-360 Joules depending upon the model and waveform used. The subcutaneous device 14 of the present disclosure uses maximum voltages in the range of about 300 to approximately 1000 Volts and is associated with energies of approximately 25 to 150 joules or more. The total high voltage capacitance could range from about 50 to about 300 microfarads. Such cardioversion-defibrillation shocks are only delivered when a malignant tachyarrhythmia, e.g., ventricular fibrillation is detected through processing of the far field cardiac ECG employing the detection algorithms as described herein below.

In FIG. 3A, sense amp 190 in conjunction with pacer/device timing circuit 178 processes the far field ECG sense signal that is developed across a particular ECG sense vector defined by a selected pair of the subcutaneous electrodes 24, 26 and 28, or, optionally, a virtual signal (i.e., a mathematical combination of two vectors) if selected. In some embodiments, sensing of ventricular depolarizations can be accomplished using an electrode 21, located on or adjacent the outer wall of the ventricle being paced. The selection of the sensing electrode pair is made through the switch matrix/MUX 191 in a manner to provide the most reliable sensing of the ECG signals of interest, which, in the present invention includes both R-waves (ventricular depolarizations) and P-waves (atrial depolarizations). The sense amp 190 thus serves as means for sensing both atrial and ventricular depolarizations.

The far field ECG signals are passed through the switch matrix/MUX 191 to and sense amplifier 190 to the pacer/device timing circuit 178, which, in conjunction with the control circuit 144 evaluates the sensed ECG signals. Bradycardia, or asystole, is typically determined by an escape interval timer within the pacer timing circuit 178 and/or the control circuit 144. Pacer/device timing circuitry 178, in conjunction with control circuitry provide means for analysis of detected atrial and ventricular depolarization waveforms and timing, for selecting the mode of therapy provided by the device and for determining the timing intervals involved in the delivery of pacing therapies.

Pace Trigger signals from pacer device/timing circuitry 178, under control of the control circuitry 144, are applied to the pacing pulse generator 192. Ventricular pacing stimulation pulses are delivered via switch matrix 191 to electrode 21, with any of the other electrodes serving as an indifferent electrode. Fusion-pacing therapy according to the disclosure is delivered via this circuitry. The pulse generator circuitry thus serves as a means for the delivery of the desired pacing therapy according to the invention. Bradycardia pacing when the interval between successive R-waves exceeds the escape interval may be provided both as part of the fusion pacing therapy according to the present invention and to maintain cardiac output after delivery of a cardioversion-defibrillation shock that may cause the heart to slowly beat as it recovers back to normal function. Sensing subcutaneous far field signals in the presence of noise may be aided by the use of appropriate denial and extensible accommodation periods as described in U.S. Pat. No. 6,236,882 “Noise Rejection for Monitoring ECGs” to Lee, et al. and incorporated herein by reference in its entirety.

Detection of a malignant tachyarrhythmia is determined in the Control circuit 144 as a function of the intervals between R-wave sense event signals that are output from the pacer/device timing 178 and sense amplifier circuit 190 to the timing and control circuit 144. and thence to the microprocessor 142. It should be noted that the present disclosure utilizes not only interval based signal analysis method but also supplemental sensors and morphology processing method and apparatus as described herein below. Analysis of morphologies of detected depolarizations for purposes of the present invention may take place in the microprocessor circuitry 142. The microprocessor circuitry comprises a means for morphology analysis, for determination of reliability of atrial sensing and detection of reverse remodeling as discussed below. In conjunction with the control circuitry 144 It also serves as a means for controlling the various switching operations between pacing therapies, for measuring time interval and for controlling the duration of the delay between detected atrial depolarizations and delivery of ventricular pacing pulses as described below.

Control circuitry 144 may take the form of a microprocessor controlled circuit as illustrated operating under a stored instruction set which defines the various operations associated with delivery of pacing therapies according to the present invention. Alternatively, fixed purpose analog or digital circuitry incorporated within the control and/or timing circuitry may perform some or all of these operations. The form of circuitry chosen is not critical to the invention so long as it is capable of performing the required operations (method steps) associated with the invention. Correspondingly, the specific division of functions between the microprocessor circuitry 142, the control circuitry 144 and the timing circuitry 178 is not critical to the invention, so long as the circuitry as a whole is capable of performing the required operations (method steps) associated with the invention

Supplemental sensors such as tissue color, tissue oxygenation, respiration, patient activity and the like may be used to contribute to the decision to apply or withhold a defibrillation therapy as described generally in U.S. Pat. No. 5,464,434 “Medical Interventional Device Responsive to Sudden Hemodynamic Change” to Alt and incorporated herein by reference in its entirety. Sensor processing block 194 provides sensor data to microprocessor 142 via data bus 146. Specifically, patient activity and/or posture may be determined by the apparatus and method as described in U.S. Pat. No. 5,593,431 “Medical Service Employing Multiple DC Accelerometers for Patient Activity and Posture Sensing and Method” to Sheldon and incorporated herein by reference in its entirety. Patient respiration may be determined by the apparatus and method as described in U.S. Pat. No. 4,567,892 “Implantable Cardiac Pacemaker” to Plicchi, et al. and incorporated herein by reference in its entirety. Patient tissue oxygenation or tissue color may be determined by the sensor apparatus and method as described in U.S. Pat. No. 5,176,137 to Erickson, et al. and incorporated herein by reference in its entirety. The oxygen sensor of the '137 patent may be located in the subcutaneous device pocket or, alternatively, located on the lead 18 to enable the sensing of contacting or near-contacting tissue oxygenation or color.

Certain steps in the performance of the detection algorithm criteria are cooperatively performed in microcomputer 142, including microprocessor, RAM and ROM, associated circuitry, and stored detection criteria that may be programmed into RAM via a telemetry interface (not shown) conventional in the art. Data and commands are exchanged between microcomputer 142 and timing and control circuit 144, pacer timing/amplifier circuit 178, and high voltage output circuit 140 via a bi-directional data/control bus 146. The pacer timing/amplifier circuit 178 and the control circuit 144 are clocked at a slow clock rate. The microcomputer 142 is normally asleep, but is awakened and operated by a fast clock by interrupts developed by each R-wave sense event, on receipt of a downlink telemetry programming instruction or upon delivery of cardiac pacing pulses to perform any necessary mathematical calculations, to perform tachycardia and fibrillation detection procedures, and to update the time intervals monitored and controlled by the timers in pacer/device timing circuitry 178.

When a malignant tachycardia is detected, high voltage capacitors 156, 158, 160, and 162 are charged to a pre-programmed voltage level by a high-voltage charging circuit 164. It is generally considered inefficient to maintain a constant charge on the high voltage output capacitors 156, 158, 160, 162. Instead, charging is initiated when control circuit 144 issues a high voltage charge command HVCHG delivered on line 145 to high voltage charge circuit 164 and charging is controlled by means of bi-directional control/data bus 166 and a feedback signal VCAP from the HV output circuit 140. High voltage output capacitors 156, 158, 160 and 162 may be of film, aluminum electrolytic or wet tantalum construction.

The negative terminal of high voltage battery 112 is directly coupled to system ground. Switch circuit 114 is normally open so that the positive terminal of high voltage battery 112 is disconnected from the positive power input of the high voltage charge circuit 164. The high voltage charge command HVCHG is also conducted via conductor 149 to the control input of switch circuit 114, and switch circuit 114 closes in response to connect positive high voltage battery voltage EXT B+ to the positive power input of high voltage charge circuit 164. Switch circuit 114 may be, for example, a field effect transistor (FET) with its source-to-drain path interrupting the EXT B+ conductor 118 and its gate receiving the HVCHG signal on conductor 145. High voltage charge circuit 164 is thereby rendered ready to begin charging the high voltage output capacitors 156, 158, 160, and 162 with charging current from high voltage battery 112.

High voltage output capacitors 156, 158, 160, and 162 may be charged to very high voltages, e.g., 300-1000V, to be discharged through the body and heart between the electrode pair of subcutaneous cardioversion-defibrillation electrodes 113 and 123. The details of the voltage charging circuitry are also not deemed to be critical with regard to practicing the present disclosure; one high voltage charging circuit believed to be suitable for the purposes of the present disclosure is disclosed. High voltage capacitors 156, 158, 160 and 162 may be charged, for example, by high voltage charge circuit 164 and a high frequency, high-voltage transformer 168 as described in detail in commonly assigned U.S. Pat. No. 4,548,209 “Energy Converter for Implantable Cardioverter” to Wielders, et al. Proper charging polarities are maintained by diodes 170, 172, 174 and 176 interconnecting the output windings of high-voltage transformer 168 and the capacitors 156, 158, 160, and 162. As noted above, the state of capacitor charge is monitored by circuitry within the high voltage output circuit 140 that provides a VCAP, feedback signal indicative of the voltage to the timing and control circuit 144. Timing and control circuit 144 terminates the high voltage charge command HVCHG when the VCAP signal matches the programmed capacitor output voltage, i.e., the cardioversion-defibrillation peak shock voltage.

Control circuit 144 then develops first and second control signals NPULSE 1 and NPULSE 2, respectively, that are applied to the high voltage output circuit 140 for triggering the delivery of cardioverting or defibrillating shocks. In particular, the NPULSE 1 signal triggers discharge of the first capacitor bank, comprising capacitors 156 and 158. The NPULSE 2 signal triggers discharge of the first capacitor bank and a second capacitor bank, comprising capacitors 160 and 162. It is possible to select between a plurality of output pulse regimes simply by modifying the number and time order of assertion of the NPULSE 1 and NPULSE 2 signals. The NPULSE 1 signals and NPULSE 2 signals may be provided sequentially, simultaneously or individually. In this way, control circuitry 144 serves to control operation of the high voltage output stage 140, which delivers high energy cardioversion-defibrillation shocks between the pair of the cardioversion-defibrillation electrodes 113 and 123 coupled to the HV-1 and COMMON output as shown in FIG. 3A.

Thus, subcutaneous device 14 monitors the patient's cardiac status and initiates the delivery of a cardioversion-defibrillation shock through the cardioversion-defibrillation electrodes 24 and 28 in response to detection of a tachyarrhythmia requiring cardioversion-defibrillation. The high HVCHG signal causes the high voltage battery 112 to be connected through the switch circuit 114 with the high voltage charge circuit 164 and the charging of output capacitors 156, 158, 160, and 162 to commence. Charging continues until the programmed charge voltage is reflected by the VCAP signal, at which point control and timing circuit 144 sets the HVCHG signal low terminating charging and opening switch circuit 114. Typically, the charging cycle takes only fifteen to twenty seconds, and occurs very infrequently. The subcutaneous device 14 can be programmed to attempt to deliver cardioversion shocks to the heart in the manners described above in timed synchrony with a detected R-wave or can be programmed or fabricated to deliver defibrillation shocks to the heart in the manners described above without attempting to synchronize the delivery to a detected R-wave. Episode data related to the detection of the tachyarrhythmia and delivery of the cardioversion-defibrillation shock can be stored in RAM for uplink telemetry transmission to an external programmer as is well known in the art to facilitate in diagnosis of the patient's cardiac state. A patient receiving the device 14 on a prophylactic basis would be instructed to report each such episode to the attending physician for further evaluation of the patient's condition and assessment for the need for implantation of a more sophisticated ICD.

Turning to FIG. 3B, the subcutaneous ECG signal (ECG1) is applied to ECG morphology block 232, filtered by a 2-pole 23 Hz low pass filter 252 and evaluated by DSP microcontroller 254 under control of program instructions stored in System Instruction RAM 258. ECG morphology is used for subsequent rhythm detection/determination (to be described herein below).

Subcutaneous device 14 desirably includes telemetry circuit (not shown in FIG. 3A), so that it is capable of being programmed by means of external programmer 20 via a 2-way telemetry link 22 (shown in FIG. 1). Uplink telemetry allows device status and diagnostic/event data to be sent to external programmer 20 for review by the patient's physician. Downlink telemetry allows the external programmer via physician control to allow the programming of device function and the optimization of the detection and therapy for a specific patient. Programmers and telemetry systems suitable for use in the practice of the present disclosure have been well known for many years. Known programmers typically communicate with an implanted device via a bidirectional radio-frequency telemetry link, so that the programmer can transmit control commands and operational parameter values to be received by the implanted device, so that the implanted device can communicate diagnostic and operational data to the programmer. Programmers believed to be suitable for the purposes of practicing the present disclosure include the Models 9790 and CareLink® programmers, commercially available from Medtronic, Inc., Minneapolis, Minn.

Various telemetry systems for providing the necessary communications channels between an external programming unit and an implanted device have been developed and are well known in the art. Telemetry systems believed to be suitable for the purposes of practicing the present disclosure are disclosed, for example, in the following U.S. patents: U.S. Pat. No. 5,127,404 to Wyborny et al. entitled “Telemetry Format for Implanted Medical Device”; U.S. Pat. No. 4,374,382 to Markowitz entitled “Marker Channel Telemetry System for a Medical Device”; and U.S. Pat. No. 4,556, 063 to Thompson et al. entitled “Telemetry System for a Medical Device”. The Wyborny et al. '404, Markowitz '382, and Thompson et al. '063 patents are commonly assigned to the assignee of the present disclosure, and are each hereby incorporated by reference herein in their respective entireties.

FIG. 3B is a schematic diagram of signal processing aspects of a subcutaneous device according to an exemplary embodiment of the present disclosure. The transthoracic ECG signal (ECG1) detected between the distal electrode 26 of subcutaneous lead 18 and one of electrodes 28 positioned on the subcutaneous device 14 are amplified and band pass filtered (2.5-105 Hz) by pre-amplifiers 202 and 206 located in Sense Amp 190 of FIG. 3A. The amplified EGM signals are directed to A/D converters 210 and 212, which operate to sample the time varying analog EGM signal and digitize the sampled points. The digital output of A/D converters 210 and 212 are applied to temporary buffers/control logic, which shifts the digital data through its stages in a FIFO manner under the control of Pacer/Device Timing block 178 of FIG. 3A. Virtual Vector block 226 selects one housing-based ECG signal (ECG2) from any pair of electrodes 28 as described, for example, in U.S. Pat. No. 5,331,966 “Subcutaneous Multi-Electrode Sensing System, Method and Pacer” to Bennett, et al. or, alternatively, generates a virtual vector signal under control of Microprocessor 142 and Control block 144 as described in U.S. Pat. No. 6,505,067 “System and Method for Deriving Virtual ECG or EGM Signal” to Lee, et al; both patents incorporated herein by reference in their entireties. ECG1 and ECG2 vector selection may be selected by the patient's physician and programmed via telemetry link 22 from programmer 20.

According to an embodiment of the present disclosure, in order to automatically select the preferred ECG vector set, it is necessary to have an index of merit upon which to rate the quality of the signal. “Quality” is defined as the signal's ability to provide accurate heart rate estimation and accurate morphological waveform separation between the patient's usual sinus rhythm and the patient's ventricular tachyarrhythmia.

Appropriate indices may include P-wave amplitude, R-wave amplitude, R-wave peak amplitude to waveform amplitude between R-waves (i.e., signal to noise ratio), low slope content, relative high versus low frequency power, mean frequency estimation, probability density function, or some combination of these metrics.

Automatic vector selection can be done at implantation or periodically (daily, weekly, monthly) or both. At implant, automatic vector selection may be initiated as part of an automatic device turn-on procedure that performs such activities as measure lead impedances and battery voltages. The device turn-on procedure may be initiated by the implanting physician (e.g., by pressing a programmer button) or, alternatively, may be initiated automatically upon automatic detection of device/lead implantation. The turn-on procedure may also use the automatic vector selection criteria to determine if ECG vector quality is adequate for the current patient and for the device and lead position, prior to suturing the subcutaneous device 14 device in place and closing the incision. Such an ECG quality indicator would allow the implanting physician to maneuver the device to a new location or orientation to improve the quality of the ECG signals as required. The preferred ECG vector or vectors may also be selected at implant as part of the device turn-on procedure. The preferred vectors might be those vectors with the indices that maximize rate estimation and detection accuracy. There may also be an a priori set of vectors that are preferred by the physician, and as long as those vectors exceed some minimum threshold, or are only slightly worse than some other more desirable vectors, the a priori preferred vectors are chosen. Certain vectors may be considered nearly identical such that they are not tested unless the a priori selected vector index falls below some predetermined threshold.

Depending upon metric power consumption and power requirements of the device, the ECG signal quality metric may be measured on the range of vectors (or alternatively, a subset) as often as desired. Data may be gathered, for example, on a minute, hourly, daily, weekly or monthly basis. More frequent measurements (e.g., every minute) may be averaged over time and used to select vectors based upon susceptibility of vectors to occasional noise, motion noise, or EMI, for example.

Alternatively, the subcutaneous device 14 may have an indicator/sensor of patient activity (piezo-resistive, accelerometer, impedance, or the like) and delay automatic vector measurement during periods of moderate or high patient activity to periods of minimal to no activity. One representative scenario may include testing/evaluating ECG vectors once daily or weekly while the patient has been determined to be asleep (using an internal clock (e.g., 2:00 am) or, alternatively, infer sleep by determining the patient's position (via a 2- or 3-axis accelerometer) and a lack of activity).

If infrequent automatic, periodic measurements are made, it may also be desirable to measure noise (e.g., muscle, motion, EMI, etc.) in the signal and postpone the vector selection measurement when the noise has subsided.

Subcutaneous device 14 may optionally have an indicator of the patient's posture (via a 2- or 3-axis accelerometer). This sensor may be used to ensure that the differences in ECG quality are not simply a result of changing posture/position. The sensor may be used to gather data in a number of postures so that ECG quality may be averaged over these postures or, alternatively, selected for a preferred posture.

In the preferred embodiment, vector quality metric calculations would occur a number of times over approximately 1 minute, once per day, for each vector. These values would be averaged for each vector over the course of one week. Averaging may consist of a moving average or recursive average depending on time weighting and memory considerations. In this example, the preferred vector(s) would be selected once per week.

Continuing with FIG. 3B, a diagnostic channel 228 receives a programmable selected ECG signal from the housing based subcutaneous electrodes and the transthoracic ECG from the distal electrode 26 on lead 18. Block 238 compresses the digital data, the data is applied to temporary buffers/control logic 218 which shifts the digital data through its stages in a FIFO manner under the control of Pacer/Device Timing block 178 of FIG. 3A, and the data is then stored in SRAM block 244 via direct memory access block 242.

The two selected ECG signals (ECG1 and ECG2) are additionally used to provide R-wave interval sensing via ECG sensing block 230. IIR notch filter block 246 provides 50/60 Hz notch filtering. A rectifier and auto-threshold block 248 provides R-wave event detection as described in U.S. Pat. No. 5,117,824 “Apparatus for Monitoring Electrical Physiologic Signals” to Keimel, et al; publication WO2004023995 “Method and Apparatus for Cardiac R-wave Sensing in a Subcutaneous ECG Waveform” to Cao, et al. and U.S. Publication No. 2004/0260350 “Automatic EGM Amplitude Measurements During Tachyarrhythmia Episodes” to Brandstetter, et al, all incorporated herein by reference in their entireties. The rectifier of block 248 performs full wave rectification on the amplified, narrowband signal from band pass filter 246. A programmable fixed threshold (percentage of peak value), a moving average or, more preferably, an auto-adjusting threshold is generated as described in the '824 patent or '350 publication. In these references, following a detected depolarization, the amplifier is automatically adjusted so that the effective sensing threshold is set to be equal to a predetermined portion of the amplitude of the sensed depolarization, and the effective sensing threshold decays thereafter to a lower or base-sensing threshold. A comparator in block 248 determines signal crossings from the rectified waveform and auto-adjusting threshold signal. A timer block 250 provides R-wave to R-wave interval timing for subsequent arrhythmia detection (to be described herein below). The heart rate estimation is derived from the last 12 R-R intervals (e.g., by a mean, trimmed mean, or median; for example); with the oldest data value being removed as a new data value is added.

FIG. 3C depicts a typical subcutaneous ECG waveform 402 and waveform 404 depicts the same waveform after filtering and rectification. A time dependant threshold 406 allows a more sensitive sensing threshold temporally with respect to the previous sensed R-wave. Sensed events 408 indicate when the rectified and filtered ECG signal 404 exceeds the auto-adjusting threshold and a sensed event has occurred.

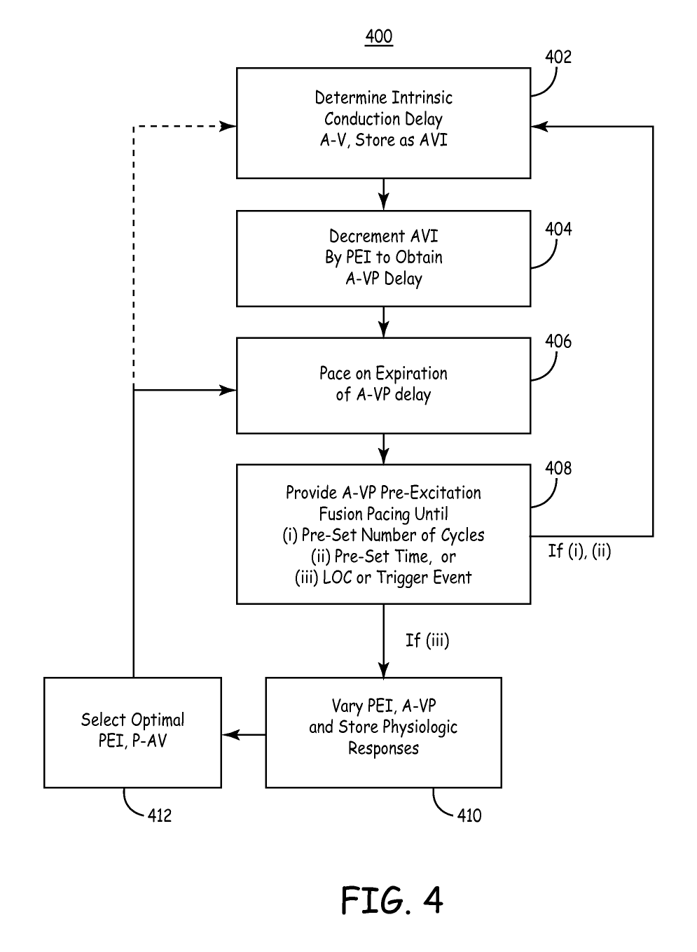

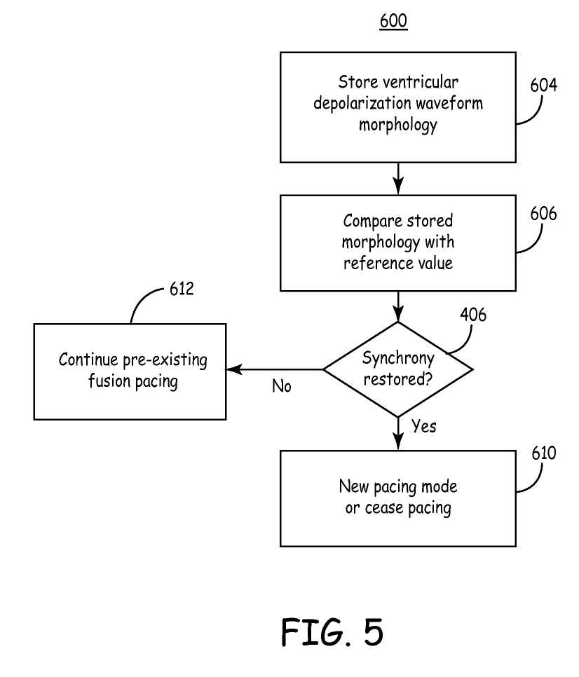

Some of the operating modes of the device circuitry of FIG. 3A are depicted in the flow charts (FIGS. 4-5) and described as follows. The particular operating mode is a programmed or hard-wired sub-set of the possible operating modes as also described below. For convenience, the algorithm of FIGS. 4-5 is described in the context of determining the PEI delay and computing A-VP intervals to optimally pace the LV chamber to produce electromechanical fusion with the corresponding intrinsic depolarization of the RV chamber. The RV chamber depolarizes intrinsically so that the pre-excited electromechanical fusion occurs as between the intrinsically activated RV chamber and the pre-excitation evoked response of the LV chamber. As noted below, the algorithm can be employed to determine an optimal PEI delay that results in an A-VP interval producing ventricular synchrony (i.e., CRT delivery via a single ventricular pacing stimulus). Of course, the methods according to the present disclosure are intended to be stored as executable instructions on any appropriate computer readable medium although they may be manually or performed by dedicated purpose analog and/or digital electronic circuitry as well.

FIG. 4 illustrates one embodiment of the present disclosure wherein the IPG circuit 300 includes a method 400 beginning with step 402 that is periodically performed to determine the intrinsic ventricular delay. In conjunction with step 402 the first-to-depolarize ventricle is understood to be the RV and the second-to-depolarize ventricle is understood to be the LV. In step 402, the device measures an A-V interval extending between an atrial depolarization sensed via the chosen pair of far-field sensing electrodes (26, 28, FIGS. 2, 3) and the following sensed ventricular depolarization. As discussed in conjunction with FIG. 3C, the ventricular depolarization is sensed when the amplitude of the filtered electrogram exceeds the detection threshold. The sensed A-V interval (AVI) is stored.

In step 404, AVI is decremented by the PEI to generate the A-VP delay for delivering pacing stimulus to the LV chamber. The magnitude of the PEI depends on several factors, including internal circuitry processing delay, location of sensing electrodes, location of pacing electrodes, heart rate, dynamic physiologic conduction status (e.g., due to ischemia, myocardial infarction, LBBB or RBBB, etc.). The inventors have found that a PEI of approximately 20-40 milliseconds (ms) oftentimes provides adequate pre-excitation to the LV chamber resulting in electromechanical fusion of both ventricles. However, a reasonable range for the PEI runs from about one ms to about 100 ms (or more).

The PEI may be fixed or variable dependent upon the sensed AVI duration, sensed heart rate (HR), a derived value combining HR with an activity sensor input, P-wave to P-wave timing, R-wave to R-wave timing and the like. PEI values may be calculated as a mathematical function of the various measured values or may be selected from a look-up table correlating desired PEI values to measured values.

Optionally, an iterative subroutine for adjusting the PEI can be used and/or a clinical procedure utilized to help define optimum values for the magnitude of the decrease in the A-VP delay. The values of PEI may be optimized, for example, based upon the waveforms of the sensed ventricular depolarizations following delivery of the pacing pulses. For example, a mechanism for varying timing of ventricular pacing pulses to minimize R-wave width as described in U.S. Pat. No. 6,804,555, issued to Warkentin and incorporated by reference in its entirety may be employed before implant to initialize the value of PEI or automatically by the device after implant to update the value of PEI as the patient's condition changes over time, as discussed in more detail below. A look-up table relating stored optimal PEI values to corresponding AVI values may thereafter be used to select the value of PEI corresponding to a sensed AVI value at step 404.

Following the decrementing step 404 the A-VP (pacing) delay interval is set and in step 406 pre-excitation pacing therapy is delivered to the LV chamber upon expiration of the A-VP interval for a defined series of cardiac cycles or for a defined time period. In the context of the atrial-synchronized ventricular fusion pacing mode described, it should also be understood that the device may also pace in the ventricle in response to the expiration of an underlying ventricular escape interval and/or in response to sensed ventricular depolarizations not preceded by associated sensed atrial depolarizations.

In the presently illustrated embodiment of the disclosure, pre-excitation pacing therapy delivery using the derived A-VP delay and PEI values continues until at step 408: (i) a pre-set number of cardiac cycles occur, (ii) a pre-set time period expires, (iii) a loss of capture occurs in the LV chamber, or another physiologic response trigger event occurs. The number of cardiac cycles or period for events (i) and (ii) may be set to any clinically appropriate value, given the patient's physiologic condition, for example Alternative indicators that the delivered fusion pacing therapy is ineffective may be used as a physiologic event response trigger. Physiological response event triggers might, for example, include excessively wide R-waves associated with delivered pacing pulses, indications if inadequate cardiac performance from an associated subcutaneous or other type of hemodynamic sensor or just the expiration of a time interval or a given number of pacing pulses greater than those associated with events (i) or (ii), respectively. If a loss of capture in the LV chamber is detected it could indicate that the ventricular pacing stimulus is being delivered too late (e.g., during the refractory period of the LV chamber) or that the LV pacing electrodes have malfunctioned or become dislodged. The pre-excitation pacing therapy could alternatively be terminated as a response to a loss of capture, under the assumption that the electrodes have become dislodged.

With respect to the physiologic response trigger step 410 an iterative process for determining appropriate PEIs may be performed. Instep 410, the current PEIs and derived A-VP delays are directly manipulated from prior operating values while one or more physiologic responses, for example R-wave widths as discussed above, are monitored and/or measured and stored. PEI values may be varied associated with various measured AVIs to derive a look up table associating the most desirable PEIs for each AVI. Such a look up table, as periodically updated, may be used at step 404 to decrement the measured AVIs to derive A-VP delays. After storing the physiologic response data (and corresponding PEIs used during data collection) at step 412 the data is compared and the PEI corresponding to the most favorable physiologic response at the current AVI is then programmed as the operating PEI. The process then proceeds back to step 406 and the LV chamber receives pre-excitation pacing therapy based on the updated, physiologically-derived PEI.

In FIG. 5, a process 600 for periodically ceasing delivery of the pre-excitation, atrial-synchronized single ventricular pacing therapy to switch to an alternative pacing therapy, or to allow normal sinus rhythm to continue chronically is illustrated. The process 600 can be implemented as a part of steps 402 or 410-412 (FIG. 4) or can be performed independently. In either case, process 600 is designed to help reveal improvement (or decline) of a patient's condition. In the former case, if so-called “reverse remodeling” of the myocardium occurs resulting in return of ventricular synchrony and improved hemodynamics and autonomic tone, pre-excitation therapy delivery may be temporarily or permanently terminated. The patient may, in the best scenario, be relieved of pacing therapy delivery altogether (programming the pacing circuitry to an ODO monitoring-only “pacing modality”). Assuming the patient is not chronotropically incompetent, normal sinus rhythm may emerge permanently for all the activities of daily living. Additionally, the process 600 may be employed to search for a change in conduction status, e.g., shortening if inter-ventricular conduction delay times. In conjunction with this process, pre-excitation pacing therapy ceases for one or more cardiac cycles and the intrinsic, normal sinus rhythm is allowed to emerge. At step 604 the morphology (e.g. width) of the intrinsic ventricular depolarization(s) is monitored and stored in memory. At step 606 an analysis of ventricular depolarization waveforms (R-waves) depolarization comparison is performed, for example by comparing the morphologies (e.g. widths) of the detected depolarization morphologies with a reference value indicative of normal ventricular synchrony. The reference value may be pre-programmed or, for example, may correspond to a best obtained result employing the pre-excitation therapy of the present invention. In the event that the intrinsic ventricular depolarization morphology indicates that return of ventricular synchrony has occurred at step 608, normal sinus rhythm is allowed to continue or a non-pre-excitation pacing therapy is initiated at step 610. Otherwise, pre-excitation pacing therapy according to the present invention may be resumed at step 612.

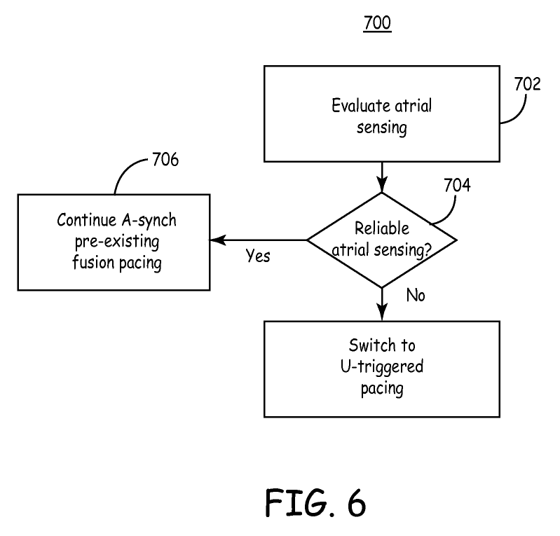

The process of FIG. 6 may be employed to switch from and atrial-synchronized pre-excitation ventricular pacing therapy to a non-atrial synchronized triggered ventricular pacing therapy responsive to loss of accurate atrial sensing. The process 700 can be implemented as a part of steps 402 or 410-412 (FIG. 4) or can be performed independently. In this aspect of the invention, the device periodically checks at step 702 to determine if a reliable pattern of atrial sensing has is ongoing. The device may, for example, evaluate the regularity of atrial sensing (regularity of the timing of sensed P-waves) and/or the frequency with which ventricular pacing pulses are generated or ventricular depolarizations (R-waves) are sensed absent prior associated atrial sensed depolarizations (P-waves). If atrial sensing is determined to be reliable at step 704, the device simply continues pacing using the atrial-synchronized pacing modality discussed above at 706. If not, the device may switch to a non-atrial-synchronized mode at 606. For example, the device may thereafter act as a triggered ventricular pacemaker (known to the art as the VVT pacing mode), stimulating the ventricle in response to either a sensed ventricular depolarization or expiration of an underlying ventricular pacing interval.

FIG. 6 illustrates an alternative embodiment of the present invention employing a subcutaneous electrode array as described in US Patent Application No. US 2006/0122649, by Ghanem, et al, as discussed above. Numbered elements correspond to identically numbered elements in FIG. 1, with the exception that a subcutaneous electrode array 721, located on subcutaneous lead 719 is substituted for electrode 21 on lead 19 of FIG. 1. Electrodes on array 721 are selected to steer stimulation energy to the left ventricle, using the techniques described in the Ghanem, et al application. The electrode array could alternately be located on the enclosure 15 of the device 14. In this embodiment, some or all of the same electrodes may be used for both pacing and sensing. Location of the electrode array in an infra-clavicular position may be desirable to reduce stimulation thresholds.

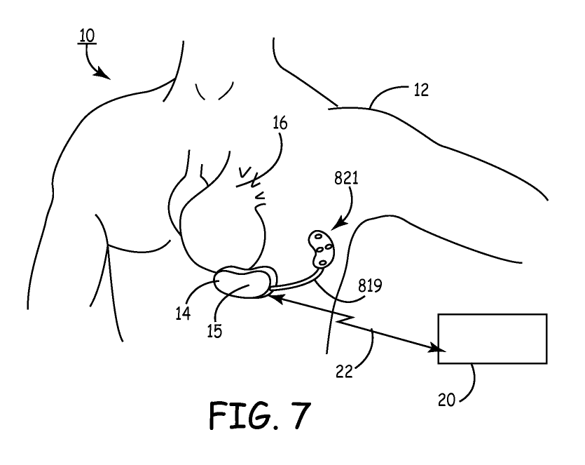

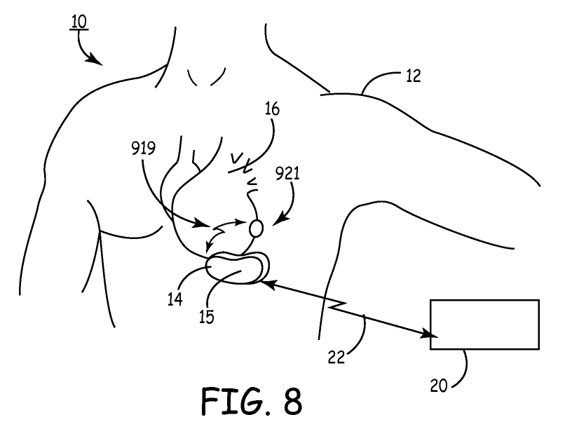

FIG. 7 illustrates an additional alternative embodiment of the present invention employing a leadless stimulation electrode array as described in U.S. Pat. No. 5,814,089, issued to Stokes, et al., as discussed above. Numbered elements correspond to identically numbered elements in FIG. 1, with the exception that a leadless electrode-bearing device 821, located on the left ventricle, is substituted for electrode 21 on lead 19 of FIG. 1. Electrodes on array 821 deliver stimulation energy to the left ventricle, using the techniques described in the Stokes, et al. patent. Stimulation is triggered by the device 14, using an RF or other communication link 819. Energy to power the pulse generation circuitry within the leadless electrode device 821 may also be transmitted using link 819 or by other mechanisms, also as disclosed in the Stokes, et al. patent.

It should be understood that, certain of the above-described structures, functions and operations of the pacing systems of the illustrated embodiments are not necessary to practice the present disclosure and are included in the description simply for completeness of an exemplary embodiment or embodiments. It will also be understood that there may be other structures, functions and operations ancillary to the typical operation of an implantable pulse generator that are not disclosed and are not necessary to the practice of the present disclosure.

1. A method of delivering a single-ventricular fusion-type pacing therapy, comprising:
a) detecting an atrial depolarization during a cardiac cycle from a far-field sensing array coupled to an extra-cardiac implantable medical device and spaced from the myocardium;
b) detecting an associated intrinsic ventricular depolarization during the cardiac cycle;
c) measuring the time elapsed between the detected P-wave and the detected R-wave;
d) decrementing the measured elapsed time interval by a predetermined amount to produce a defined delay;
e) initiating the defined delay responsive to subsequent sensed atrial depolarizations; and
f) delivering a pacing stimulus to a single ventricle via a non-transvenous electrode at expirations of the defined delays.

2. A method according to claim 2, wherein the decrementing amount is between about 10 milliseconds (ms) and about 50 ms.

3. A method according to claim 1, wherein detecting the R-wave comprises sensing the R-wave using the far-field sensing array.

4. A method according to claim 4, wherein detecting the R-wave comprises sensing the R-wave using a myocardial or pericardial electrode.

5. A method according to claim 1, further comprising:
detecting an arrhythmia via one of the far-field sensing array, an epicardial patch-type assembly, a pericardial electrode, and a helical screw-type electrode;
ceasing delivery of the single-ventricular fusion-pacing therapy; and
delivering one of an anti-tachycardia pacing sequence and a defibrillation therapy.

6. A method according to claim 5, wherein detecting the arrhythmia comprises detecting a ventricular arrhythmia.

7. A method according to claim 1, further comprising detecting a failure to reliably sense P-waves and in response thereto switching to an R-wave triggered pacing therapy.

8. A method according to claim 7, wherein detecting a failure to reliably sense P-waves comprises detecting R-waves or delivered pacing pulses without associated preceding detected P-waves.

9. A method according to claim 7, wherein detecting a failure to reliably sense P-waves comprises determining the regularity of timing of sensed P-waves

10. A method according to claim 1, further comprising detecting reverse ventricular remodeling and in response thereto switching to an alternate pacing therapy.

11. A method according to claim 1, further comprising detecting reverse ventricular remodeling and in response thereto allowing normal, un-paced sinus rhythm to resume.

12. A method according to claim 9, wherein detecting reverse ventricular remodeling comprises analysis of morphologies of detected R-waves.

13. A method according to claim 1, further comprising analysis of morphologies of detected R-waves and variation of the defined delay in response thereto.

14. A method according to claim 13, further comprising analysis of morphologies of detected R-waves comprises measuring widths of the detected R-waves.

15. A method according to claim 13, wherein analysis of morphologies of detected R- waves comprises analysis of morphologies of detected R- waves sensed using the far-field sensing array.

16. An apparatus for establishing a fusion pacing delay interval for a cardiac therapy device, comprising:
a) means for detecting an atrial depolarization during a cardiac cycle comprising a far-field sensing array coupled to an implantable medical device and spaced from the myocardium;
b) means for detecting an intrinsic ventricular depolarization during the cardiac cycle;
c) means for measuring the time interval elapsed between the detected atrial depolarization and the detected ventricular depolarization;
d) means for decrementing the measured interval by a predetermined amount to produce a defined delay;
e) means for timing the defined delay following a sensed atrial depolarization; and
f) means for delivering a pacing stimulus to a single ventricle using an electrode located exterior to the heart at the expiration of the defined delay.

17. An apparatus according to claim 16, wherein the predetermined decrementing amount is an interval of between about 10 milliseconds (ms) and about 50 ms.

18. An apparatus according to claim 16, wherein the electrode comprises one of an epicardial electrode, a myocardial electrode, a pericardial electrode, a subcutaneous electrode.

19. An apparatus according to claim 16, wherein the means for detecting the R-wave comprises one of an epicardial electrode, a myocardial electrode, a pericardial electrode, a subcutaneous electrode.

20. An apparatus according to claim 16, further comprising: means for detecting an arrhythmia;
means for ceasing delivery of the single-ventricular fusion-pacing therapy; and
means responsive to detection of an arrhythmia for delivering one of an anti-tachycardia pacing (ATP) sequence and a defibrillation therapy.

21. An apparatus according to claim 20, wherein the means for detecting an arrhythmia comprises means for detecting a ventricular arrhythmia.

22. An apparatus according to claim 16, further comprising means for detecting a failure to reliably sense P-waves and in response thereto switching to an R-wave triggered pacing therapy.

23. An apparatus according to claim 22, wherein the means for detecting a failure to reliably sense P-waves comprises means for detecting R-waves or delivered pacing pulses without associated preceding detected P-waves.

24. An apparatus according to claim 22, wherein detecting a failure to reliably sense P-waves comprises determining the regularity of timing of sensed P-waves

25. An apparatus according to claim 16, further comprising means for detecting reverse ventricular remodeling and in response thereto switching to an alternate pacing therapy.

26. An apparatus according to claim 16, further comprising means for detecting reverse ventricular remodeling and in response thereto allowing normal, un-paced sinus rhythm to resume.

27. An apparatus according to claim 26, wherein the means for detecting reverse ventricular remodeling comprises means for analysis of morphologies of detected R-waves.

28. An apparatus according to claim 16, further comprising means for analysis of morphologies of detected R-waves and variation of the defined delay in response thereto.

29. An apparatus according to claim 28, wherein the means for analysis of morphologies of detected R-waves comprises means for measuring widths of the detected R-waves.

30. An apparatus according to claim 29, wherein analysis of morphologies of detected R-waves comprises analysis of morphologies of detected R-waves sensed using the far-field sensing array.

31. A cardiac therapy apparatus for delivering single-ventricular fusion pacing therapy, comprising:
a) a P-wave detecting circuit coupled to a far-field subcutaneous electrode array;
b) an R-wave detecting circuit coupled to the subcutaneous electrode array;
c) a timer measuring the time elapsed between a detected P-wave and a detected R-wave;
d) means for decrementing the measured elapsed time by a predetermined amount to determine a defined delay;
e) a pacing timer timing the defined delay following a sensed P-wave; and
f) a pacing pulse generator coupled to a non-transvenous electrode, delivering a pacing pulse responsive to expiration of the defined delay.

32. A cardiac therapy device according to claim 31 wherein the non-transvenous electrode comprises one of an epicardial electrode, a myocardial electrode, a pericardial electrode, a subcutaneous electrode.
